(19) United States
(12) Patent Application Publication
(10) Pub. No.: US 2015/0050373 A1
(43) Pub. Date: Feb. 19, 2015

(54) HERBAL COMPOSITION FOR THE TREATMENT OF METABOLIC DISORDERS

(71) Applicant: PIRAMAL ENTERPRISES LIMITED, Mumbai (IN)

(72) Inventors: Arvind Saklani, Mumbai (IN); Parikshit Gaikwad, Mumbai (IN); Aslam Burhan, Mumbai (IN); Somesh Sharma, Los Altos, CA (US)

(73) Assignee: PIRAMAL ENTERPRISES LIMITED, Mumbai (IN)

(21) Appl. No.: 14/381,062

(22) PCT Filed: Mar. 12, 2013
(86) PCT No.: PCT/IB2013/051925

§ 371 Date: Aug. 26, 2014

Related U.S. Application Data
(60) Provisional application No. 61/610,002, filed on Mar. 13, 2012.

(51) Int. Cl. A61K 36/185

(52) U.S. Cl. CPC A61K 36/185
USPC 424/775

(57) ABSTRACT

The present invention relates to an herbal composition comprising a therapeutically effective amount of the extract of a plant belonging to Calophyllum species as an active ingredient and optionally, a pharmaceutically acceptable carrier. The present invention also relates to herbal composition comprising extract obtained from the plant Calophyllum inophyllum. The invention also relates to a process for the preparation of the extract. The invention also relates to a method for the treatment of metabolic disorders using the said composition. The present invention also relates to a composition comprising a therapeutically effective amount of extract of the plants from Calophyllum species in combination with a known therapeutically active agent for use in the treatment of metabolic disorders.

### FIELD OF THE INVENTION

The present invention relates to a herbal composition comprising a therapeutically effective amount of the extract of a plant belonging to Calophyllum species as an active ingredient either alone, or with a pharmaceutically acceptable carrier. The composition of the present invention is useful for the treatment of metabolic disorders. The present invention also relates to a process for the manufacture of the herbal composition.

### BACKGROUND OF THE INVENTION

Metabolic disorders are the disorders or defects that occur when the body is unable to properly metabolise carbohydrates, lipids, proteins, or nucleic acids. Most metabolic disorders are caused by genetic mutations that result in missing or dysfunctional enzymes that are needed for the cell to perform metabolic processes. Examples of metabolic disorders include obesity, excessive body fat, hyperlipidemia, hyperlipoproteinemia, hyperglycemia, hypercholesterolemia, hyperinsulinemia, insulin resistance, glucose intolerance, and diabetes mellitus particularly type 2 diabetes.

Diabetes mellitus is a metabolic disorder that affects the ability to produce or use insulin in an individual. Blood glucose levels are higher than normal for individuals with diabetes. Uncontrolled diabetes is the leading cause of blindness, renal failure, non-traumatic limb amputation and premature cardiovascular mortality. Diabetic patients are also at an increased risk of developing cardiovascular disease events due to risk factors such as dyslipidemia, obesity, hypertension and glucose intolerance.

People with type 2 diabetes require regular monitoring of blood glucose levels and continuing treatment to maintain normal or near normal blood glucose levels. Treatment of NIDDM includes lifestyle adjustments, self-care measures and medicines, which can minimise the risk of diabetes and diabetes related cardiovascular complications. A number of medicines are available to treat NIDDM which include metformin; sulfonyl ureas such as glipizide; GLP antagonists such as exenatide and liraglutide; thiazolidinediones such as pioglitazone and rosigiitazone; DPP-IV inhibitors such as sitagliptin, saxagliptin, and vildagliptin; and alpha-glucosidase inhibitors such as acarbose and miglitol.

Another prevalent metabolic disorder is obesity which primarily results from an imbalance between energy intake and expenditure, A positive energy balance resulting from a chronic disparity between the intake of energy and its expenditure leads to weight gain and eventually obesity. Although it is not generally a life-threatening disease, obesity is becoming a major health problem worldwide. Obesity amplifies the risks of hypertension, dyslipidaemia, type 2 diabetes, cardiovascular disease, obstructive sleep apnoea, osteoarthritis, and several cancers. The most common approach to overcome obesity is to bring about changes in lifestyle, specifically dieting and exercise. However, achieving significant weight loss and maintaining a lower body weight in the long run is difficult. Further, obesity can also be controlled by means of drugs. Despite promising results on body weight reduction and some cardiovascular risk factors, most anti-obesity drugs developed so far have not been approved or have had to be withdrawn from the market, due to adverse side effects. As sibutramine is no longer available, orlistat is currently the only anti-obesity drug to have been approved for long-term use. Thus, the need for the development of a safe as well as effective therapy for the treatment of metabolic disorders such as NIDDM and/or obesity is still needed.

In order to select and develop new drug candidates for the treatment of metabolic disorders, two novel enzyme targets, diacylglycerol acyltransferase-1 (DGAT-1) and stearoyl-CoA desaturase-1. (SCD-1) can be utilised. These enzymes play a key role in the synthesis of triglyceride, the main form in which energy is stored in the body.

DGAT-1 is an endoplasmic membrane-bound enzyme that catalyses the biosynthesis of triglyceride at the final step of the process, converting diacylglycerol (DAG) and fatty acyl-coenzyme A (CoA) into triglyceride. The enzymatic activity is present in all cell types because of the necessity of producing triglyceride for cellular needs. DGAT-1 is highly expressed in the intestine and adipose with lower levels in the liver and muscle. Inhibition of DGAT-1 in each of these tissues (intestine, adipose, liver and muscle) would inhibit triacylglycerol synthesis and may reverse the pathophysiology of excessive lipid accumulation in human metabolic disease (Expert Opin. Ther. Patents 17(11), 1331-1339,(2007)).

Stearoyl-CoA Desaturase-1 (SCD-1), has been described as one of the major enzymes in the control of lipid metabolism and may represent a potential new therapeutic target. SCD-1 is a rate-limiting enzyme that catalyzes the biosynthesis of monounsaturated fatty acids from saturated fatty acids. The preferred substrates of SCD-1, stearate (C18:0) and palmitate (C16:0), are converted to oleate (C18:1) and palmitoyleate (C16:1) respectively. These monounsaturated fatty acids are considered as the major components of various lipids including triglycerides, cholesteryl esters, phospholipids and wax esters. Studies in experimental animals suggest that inhibiting or reducing the activity of these enzymes results in resistance to development of obesity, diabetes and associated complications (European Journal of Pharmacology, 618, 28-36, (2009), European Journal of Pharmacology, 650, 663-672, (2011)).

In the modern era of medicine, herbal materials and plants continue to play an important role in drug discovery and development. Natural products offer large structural diversity. Availability of modern techniques for separation, structure elucidation, screening and combinatorial synthesis, has led to revitalization of plant products as sources of new drugs. The introduction of herbals in the form of nutraceuticals and dietary supplements are also changing the plant-based drug market. Natural products and their analogs can be developed into useful drug candidates (Pharmacol. Res., 60(3): 195-206, (2008); Drug Discov. Today, 13(3-4), 161-71, (2009)). The demand for plant-based medicines is ever growing as crude or processed products from plants are believed to have minimum or no adverse effects as compared to the synthetic small molecules.

Calophyllum is a flowering plant genus of around 180-200 species of tropical evergreen trees. The Calophyllum species consists of four subcategories which include Calophyllum brasiliense, Calophyllum caledonicurn, Calophyllum inophyllum and Calophyllum soulattri.

Calophyllum inophyllum, is a medium to large sized evergreen tree, that averages 25-65 feet in height with a broad spreading crown of irregular branches. It is native to East Africa, India, South East Asia, Australia, South Pacific and Hawaiian islands. Different medicinal uses of this plant have been reported in the literature, for example, decoction of the bark of this plant is repotted to be used in internal hemorrhages and as a wash for indolent ulcers. Further, oil obtained from the nuts of this plant is traditionally used for medicine and cosmetics. The oil extracted from the seeds Calophyllum inophyllum is used in rheumatoid arthritis or joint disorders; itching; eczema; pimples appearing on head; eye diseases; and kidney failure. (Dravyaguna-Vij\check{n}ñna. Chaukhambha Bharati Academy, Publisher and distributor of monumental treatise of the east, Varanasi, India, Vol. II, 787, (2003); Chakradatta of Sri Chakrapanidatta. Dwivedy R. (ed.) Chaukhambha Sanskrit sansthan, Publishers and distributors of oriental cultural literature, Varanasi, India, pages 280, 354 and 499, (2002)).

It has been indicated herein above that considering the growing prevalence of metabolic disorders such as type 2 diabetes and obesity, there continues to be a need for new compositions and methods for the effective treatment of the metabolic disorders. In fact, efforts of the inventors of the present invention directed to find, a solution to these problems have resulted in an herbal composition comprising an extract of a plant, belonging to the Calophyllum species, having DGAT-1 and SCD-1 inhibitory activity, and hence is useful for the treatment, of metabolic disorders.

### SUMMARY OF THE INVENTION

According to one aspect of the present invention, there is provided a composition comprising a therapeutically effective amount of an extract of a plant, belonging to Calophyllum species as an active ingredient, and optionally at least one pharmaceutically acceptable carrier, for use in the treatment of a metabolic disorder.

According to one aspect of the present invention, there is provided a composition comprising a therapeutically effective amount of an extract of a plant selected from Calophyllum brasiliense, Calophyllum caledonicum, Calophyllum inophyllum and Calophyllum soulattri, as an active ingredient and optionally at least one pharmaceutically acceptable carrier, for use in the treatment of metabolic disorder.

According to another aspect of the present invention, there is provided a composition comprising a therapeutically effective amount of extract of the plant, from Calophyllum species, for use in combination with a therapeutically active agent, and at least one pharmaceutically acceptable carrier, for the treatment of a metabolic disorder.

According to yet another aspect of the present invention, there is provided a composition comprising a therapeutically effective amount of an extract of the plant, Calophyllum inophyllum, as an active ingredient and at least one pharmaceutically acceptable carrier, for use in the treatment of a metabolic disorder.

In another further aspect, the present invention is directed to a method for the treatment of a metabolic disorder in a subject comprising administering to a the subject a composition comprising a therapeutically effective amount of an extract of a plant, belonging to Calophyllum species as an active ingredient and optionally at least one pharmaceutically acceptable carrier.

According to another aspect of the present invention, there is provided a process for the preparation of the composition, comprising a therapeutically effective amount of extract of the plant from Calophyllum species.

### DETAILED DESCRIPTION OF THE INVENTION

It should be understood that the detailed description and specific examples, while indicating embodiments of the invention, are given by way of illustration only, since various changes and modifications within the spirit and scope of the invention will become apparent to those skilled in the art. One skilled in the art, based upon the description herein, may utilize the present invention to its fullest extent. The following specific embodiments are to be construed as merely illustrative, and not limitative of the remainder of the disclosure in any way whatsoever.

Unless defined otherwise, all technical and scientific terms used herein have the same meaning as commonly understood by one of ordinary skill in the art to which the invention belongs.

It should he noted that, as used in the specification and the appended claims, the singular forms “a,” “an,” and “the” include plural referents unless the content clearly dictates otherwise.

The term “metabolic disorder” refers to the disorders or defects that occur when the body is unable to properly metabolise carbohydrates, lipids, proteins, or nucleic acids. The metabolic disorder includes insulin resistance, hyperglycemia, type 2 diabetes, obesity, glucose intolerance, hypercholesterolemia, dyslipidemia, hyperinsulinemia, atherosclerotic disease, polycystic ovary syndrome, coronary artery disease, metabolic syndrome, hypertension, or a related disorder associated with abnormal plasma lipoprotein, triglycerides or a disorder related to glucose levels such as pancreatic beta cell regeneration.

The term “treating”, “treat” or “treatment” as used herein includes preventive (prophylactic) and palliative treatment.

The term “pharmaceutically acceptable” as used herein means the carrier, diluent, and/or excipients used in the composition must be compatible with the other ingredients of the formulation, and not deleterious to the recipient thereof.

Calophyllum is a flowering plant genus of around 180-200 species of tropical evergreen trees. The Calophyllum species consists of four subcategories which include Calophyllum brasiliense, Calophyllum caledonicum, Calophyllum inophyllum and Calophyllum soulattri. The term “Calophyllum” is intended to include all its synonyms.

The terms “herbal composition” or “composition” are used interchangeably and may refer to a composition comprising therapeutically effective amount of the extract of a plant belonging to Calophyllum species either alone or with at least one pharmaceutically acceptable carrier or excipient. The term “either alone” may further indicate that the composition contains only the extract of a plant belonging to Calophyllum species without any pharmaceutically acceptable carrier added therein. It should be noted that the term “composition” should be construed in a broad sense and includes any composition which is intended for the purpose of achieving a therapeutic effect whether sold as a pharmaceutical product, for example carrying a label as to the intended indication, whether sold over the counter, or whether sold as a phytopharmaceutical.

The term “pharmaceutically acceptable carrier” as used herein means a non-toxic, inert solid, semi-solid, diluent, encapsulating material or formulation auxiliary of any type. Some examples of materials which can serve as pharmaceutically acceptable carriers are sugars such as lactose, glucose, and sucrose; starches such as corn starch and potato starch; cellulose and its derivatives such as sodium carboxymethyl cellulose, ethyl cellulose and cellulose acetate; malt; gelatin; as well as other non-toxic compatible lubricants such as sodium lauryl sulfate and magnesium stearate, as well as coloring agents, releasing agents, coating agents, sweetening, flavoring and perfuming agents; preservatives and antioxidants can also be used in the composition, according to the judgment of the formulator.

The term “therapeutically effective amount” as used herein means an amount of the extract (e.g., the “Calophyllum inophyllum” extract) or the composition containing the extract, which is sufficient to significantly induce a positive modification in the condition to be regulated or treated, but low enough to avoid side effects if any (at a reasonable benefit/risk ratio), within the scope of sound medical judgment. The therapeutically effective amount of the extract or composition will vary with the particular condition being treated e.g. type 2 diabetes or obesity, the age and physical condition of the end user, the severity of the condition being treated/prevented, the duration of the treatment, the nature of concurrent therapy, the particular pharmaceutically acceptable carrier utilized, and like factors. As used herein, all percentages are by weight unless otherwise specified.

The term “Calophyllum inophyllum extract” or “the extract of Calophyllum inophyllum” as used herein means a blend of compounds present in any part of the plant Calophyllum inophyllum. Such compounds can be extracted from any part of the plant, such as the bark, twig, stem and wood of the plant, using extraction procedures well known in the art e.g., by carrying out the extraction procedure using organic solvents such as lower alcohols e.g. methanol or ethanol, alkyl esters such as ethyl acetate, alkyl ethers such as diethyl ether, alkyl ketones such as acetone, chloroform, petroleum ether, hexane and/or aqueous solvents such as water. The plant material can also he extracted by using mixture of solvents in a suitable ratio such as hexane-ethyl acetate (1:1), chloroform-methanol (1:1) or methanol-water (3:1).

The term “subject” as used herein refers to an animal, particularly a mammal, and more particularly a human.

The term “mammal” used herein refers to warm-blooded vertebrate animals of the class Mammalian, including humans, characterized by a covering of hair on the skin and, in the female, milk-producing mammary glands for nourishing the young. The term mammal includes animals such as cat, dog, rabbit, bear, fox, wolf, monkey, deer, mouse, pig and the human.

In an embodiment, the process for the preparation of “Calophyllum inophyllum extract” involves use of methanol as the solvent. For example, the extract can be obtained by extraction of pulverized bark of the plant Calophyllum inophyllum using methanol as the solvent.

In an embodiment, the pulverized bark of the plant Calophyllum inophyllum can be extracted using methanol-water mixture in different ratios, e. g. methanol-water (9:1) mixture, methanol-water (3:1) mixture or methanol-water (1:1) mixture can be used for extraction.

The process for preparation of the extract of the plant Calophyllum inophyllum can be easily scaled up for large-scale preparation.

“Calophyllum inophyllum extract” can be standardized using conventional techniques such as high performance liquid chromatography (HPLC) or high performance thin-layer chromatography (HPTLC). The term “standardized extract” refers to an extract which is standardized by identifying characteristic bioactive ingredient(s) or bioactive marker (s) present in the extract.

The term “active ingredient” or “bioactive ingredient” as used herein refers to “Calophyllum inophyllum extract” containing one or more bioactive compounds (bioactive markers). Bioactive ingredients can be identified using various techniques such as high performance thin-layer chromatography (HPTLC) or high performance liquid chromatography (HPLC). Bioactive markers can be isolated from the extract of the plant Calophyllum inophyllum by bioactivity guided column chromatographic purification and preparative high performance liquid chromatography (HPLC). Compounds may be characterized by analysis of the spectral data.

The term “bioactive marker” is used herein to define a characteristic (or a phytochemical profile) of an active compound which is correlated with an acceptable degree of pharmaceutical activity. “Bioactive marker”, which is the active compound, may be isolated from the extract obtained from the plant, Calophyllum inophyllum by bioactivity guided column chromatographic purification and preparative HPLC. The isolated compounds (bioactive marker) may be characterized by analysis of the spectral data.

The biological activity determination of the extracts can be carried out using various well-known biological in vitro and in vivo assays. For example, preliminary in vitro activity determination of the extracts can be carried out using assays such as diacyl glycerolacyitransferase-1 (DGAT-1) assay, stearoyl-CoA Desaturase-1 (SCD-1) assay or triglyceride synthesis assay. The in vivo activity can be determined by using assays such as the high fat diet (HFD) induced obesity model.

In an embodiment, the invention provides an herbal composition comprising a therapeutically effective amount of an extract of the plant Calophyllum inophyllum and optionally at least one pharmaceutically acceptable carrier.

In another embodiment, the invention relates to an herbal composition comprising standardized extract of the plant Calophyllum inophyllum and optionally, at least a pharmaceutically acceptable carrier.

The herbal composition of the present invention comprises 5-100% of the extract of the plant Calophyllum inophyllum.

The herbal composition of the present invention comprises 5-100% of the extract, obtained from the plant Calophyllum inophyllum containing at least one bioactive marker.

In an embodiment, the invention provides the use of the composition comprising a therapeutically effective amount of the extract of the plant Calophyllum inophyllum, for the manufacture of a medicament for the treatment of metabolic disorders.

The “Calophyllum inophyllum extract” is mixed with pharmaceutically acceptable carriers and formulated into therapeutic dosage forms.

The compositions comprising a therapeutically effective amount of the extract of the plant Calophyllum inophyllum can be administered orally, for example in the form of pills, tablets, coated tablets, capsules, powders, granules, elixirs or syrup.

The oral compositions, containing 5-100% by weight of the “Calophyllum inophyllum extract” can be prepared by thoroughly mixing the extract with pharmaceutically acceptable carrier/s, by using conventional methods.

In an embodiment, the said compositions are provided for the treatment of metabolic disorders.

In an embodiment the said compositions are provided for the treatment of metabolic disorders selected from: type 2 diabetes, obesity, glucose intolerance, hypercholesterolemia, dyslipidemia, hyperinsulinemia, atherosclerotic disease, polycystic ovary syndrome, coronary artery disease, metabolic syndrome, or hypertension.

In an embodiment the said composition is provided for the treatment of type 2 diabetes.

In an embodiment the said composition is provided for the treatment of obesity.

In an embodiment the said composition is provided for the treatment of dyslipidemia.

In an embodiment the said compositions are provided for the treatment of metabolic disorders related to disorders associated with abnormal plasma lipoprotein, triglycerides.

In an embodiment the said compositions are provided for the treatment of metabolic disorders related to glucose levels such as pancreatic beta cell regeneration.

In yet another embodiment, the present invention relates to a composition comprising a therapeutically effective amount of an extract of the plants from Calophyllum species, for use in combination with a therapeutically active agent, and at least a pharmaceutically acceptable carrier, for use in the treatment of a metabolic disorder.

In yet another embodiment, the present invention relates to a composition comprising a therapeutically effective amount of the extract of the plant Calophyllum inophyllum and optionally, at least a pharmaceutically acceptable carrier, for use in combination with a therapeutically active agent, for use in the treatment of a metabolic disorder.

In yet another embodiment, the composition of the present invention comprising a therapeutically effective amount of the extract of the plant Calophyllum inophyllum, may optionally be used in combination with a therapeutically active agent, for use in the treatment of a metabolic disorder.

The therapeutically active agent may be selected from the known bioactive substances such as orlistat, pioglitazone, rosiglitazone, glibenclamide, glipizide, glimeperide, repaglinide, nateglinide, or metformin.

The present invention is also related to a method of treating a metabolic disorder comprising the administration of the composition comprising a therapeutically effective amount of the extract of the plant Calophyllum inophyllum and optionally, at least a pharmaceutically acceptable carrier, selectively by oral route.

The herbal composition of the present invention may be formulated for oral administration by compounding the active ingredient i.e. the extract with the usual non-toxic pharmaceutically acceptable carrier/s for powders, pills, tablets, coated tablets, pellets, granules, capsules, solutions, emulsions, suspensions, elixirs, syrup, and any other form suitable for use. Formulations of the present invention encompass those which include talc, water, glucose, lactose, sucrose, gum acacia, gelatin, mannitol, starch paste, magnesium trisilicate, corn starch, keratin, colloidal silica, potato starch, urea, and cellulose and its derivatives such as sodium carboxymethyl cellulose, ethyl cellulose and cellulose acetate; malt; gelatin; as well as other non-toxic compatible lubricants such as sodium lauryl sulfate and magnesium stearate, releasing agents, coating agents and other excipients suitable for use in manufacturing preparations, in solid, semisolid or liquid form and in addition auxiliary, stabilizing, thickening and coloring agents may be used. For preparing solid compositions such as tablets or capsules, the extract is mixed with a pharmaceutical carrier (e.g., conventional tableting ingredients such as corn starch, lactose, sucrose, sorbitol, talc, stearic acid, magnesium stearate, dicalcium phosphate or gums) and other pharmaceutical diluents (e.g., water) to form a solid composition. This solid composition is then subdivided into unit dosage forms containing an effective amount of the composition of the present invention. The tablets or pills containing the extract can be coated or otherwise compounded to provide a dosage form affording the advantage of prolonged action.

The liquid forms, in which the extract may be incorporated for administration orally or by injection, include aqueous solution, suitably flavored syrups, aqueous or oil suspensions, and flavored emulsions with edible oils as well as elixirs and similar pharmaceutical vehicles. Suitable dispersing or suspending agents for aqueous suspensions include synthetic natural gums, such as fragacanth, acacia, alginate, dextran, sodium carboxymethyl cellulose, methylcellulose, polyvinylpyrrolidone or gelatin. Liquid preparations for oral administration may take the form of, for example, solutions, syrups or suspensions, or they may be presented as a dry product for reconstitution with water or other suitable vehicles before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); preservatives (e.g., methyl or propyl p-hydroxybenzoates or sorbic acid); and artificial or natural colors and/or sweeteners.

The selected dosage level will depend upon a variety of factors including the activity of the particular extract of the present invention employed, the route of administration, the time of administration, the rate of excretion of the particular composition being employed, the duration of the treatment, used in combination with the other extracts, the age, sex, weight, condition, general health and prior medical history of the patient being treated, and like factors well known in the medical arts. In general, however, doses employed for adult human treatment will typically be in the range of 0.02-5000 mg per day or 1-1500 mg per day. The desired dose may conveniently be presented in a single dose or as divided doses administered at appropriate intervals, for example as two, three, four or more sub-doses per day.

The present invention will be more readily understood by referring to the following examples which are given to illustrate the invention but do not limit its scope.

### EXAMPLES

The following terms/abbreviations are employed in the examples:

| | |
|---|---|
| L: Litre | dig: Microgram |
| mL: Millilitre | w/v: Weight by volume |
| μL: Microlitre | h: Hours |
| g: Gram | min: Minutes |
| mg: Milligram | RT: Room Temperature (25 ± 5° C.) |

### Extractions of the Plant

The plant materials (bark, wood, stem and twig) of Calophyllum inophyllum were collected from Mumbai, Maharashtra, India. A microscopic and macroscopic study for authentication was carried out for each plant material. A specimen for each part of the plant Calophyllum inophyllum has been retained in Botany Department, Piramal Healthcare Limited, Goregaon, Mumbai, Maharashtra, India.

The plant materials (hark, wood, stem and twig) of Calophyllum inophyllum were chopped into small pieces and were dried with the help of dehumidifier. The completely dried material was then coarsely ground using a pulveriser.

### Example 1

Dried, pulverized plant material of Calophyllum inophyllum (bark) (500 g) was extracted using methanol (4 L) by stirring at 45° C. for 3 h. This extraction process was repeated twice with methanol (3.5 L). The extracts were combined and concentrated to dryness. Yield: 115 g (23%).

Extract so obtained in Example 1 is referred herein as “Extract of Example 1”.

### Example 2

Dried, pulverized plant material of Calophyllum inophyllum (bark) (100 g) was extracted using methanol:water (9:1) (900 mL) by stirring at 45° C. for 3 h. This extraction process was repeated twice with methanol:water (9:1) (700 mL). The extracts were combined and concentrated. The concentrated material was lyophilized by using freeze-dryer (Edwards). Yield: 17.72 g (17.7%).

Extract so obtained in Example 2 is referred herein as “Extract of Example 2”.

### Example 3

Dried, pulverized plant material of Calophyllum inophyllum (bark) (50 g) was extracted using methanol:water (3:1) (500 mL) by stirring at 45° C. for 3 h. This extraction process was repeated twice with methanol:water (3:1) (400 mL). The extracts were combined and concentrated. The concentrated material was lyophilized by using freeze-dryer (Edwards). Yield: 8.2 g (16.4%).

Extract so obtained in Example 3 is referred herein as “Extract of Example 3”.

### Example 4

Dried, pulverized plant material of Calophyllum inophyllum (bark) (50 g) was extracted using methanol:water (1:1) (500 mL) by stirring at 45° C. for 3 h. This extraction process was repeated twice with methanol:water (1:1) (400 mL). The extracts were combined and concentrated. The concentrated material was lyophilized by using freeze-dryer (Edwards). Yield: 6.3 g (12.6%).

Extract so obtained in Example 4 is referred herein as “Extract of Example 4”.

### Example 5

Dried, pulverized plant material of Calophyllum inophyllum (stem) was extracted using methanol (1:10 w/v) by stirring at 45° C. for 3 h. The extract was filtered. This extraction process was repeated twice with methanol (1:8 w/v). The extracts were combined and concentrated. Yield: 6.5%.

Extract so obtained in Example 5 is referred herein as “Extract of Example 5”.

### Example 6

Dried, pulverized plant material of Calophyllum inophyllum (twig) was extracted using methanol (1:10 w/v) by stirring at 45° C. for 3 h. This extraction process was repeated twice with methanol (1:8 w/v). The extracts were combined and concentrated to dryness. Yield: 10%.

Extract so obtained in Example 6 is referred herein as “Extract of Example 6”.

### Example 7

Dried, pulverized plant material of Calophyllum inophyllum (wood) was extracted using methanol (1:10 w/v) by stirring at 45° C. for 3 h. This extraction process was repeated twice with methanol (1:8 w/v). The extracts were combined and concentrated to dryness. Yield: 10.6%.

Extract so obtained in Example 7 is referred herein as “Extract of Example 7”.

Extract of Example 1 to Extract of Example 7 were stored polypropylene vial in cold room at 4° C. to 8° C.,

### Pharmacological Assays

The efficacy of the extract of the plant, Calophyllum inophyllum in inhibiting the activity of DGAT-1 and SCD-1 enzymes was determined by a number of pharmacological assays, well known in the art and described below.

The following terms/abbreviations are employed in the examples:

| | |
|---|---|
| DMSO: Dimethyl sulfoxide | mM: Millimolar |
| NaOH: Sodium hydroxide | M: Molar |
| MgCl₂: Magnesium chloride | mg/kg: Milligram per kilogram |
| KCl: Potassium chloride | μg/mL: Microgram per millilitre |
| ng/μL: Nanogram per microlitre | BSA: Bovine Serum Albumin |
| DAB: DGAT Assay Buffer | FBS: Fetal Bovine Serum |
| PBS: Phosphate Buffered Saline | ORE: Open Reading Frame |
| pfu: Plaque forming units | dpm: Disintegrations per minute |
| cpm: Counts per minute | rpm: Revolutions per minute |
| MOI: Multiplicity of infection | RZPD: German Resource Center |

K₂HPO₄: Potassium dihydrogen phosphate
Na₂H₂PO₄•2H₂O: Sodium dihydrogen phosphate dihydrate
EDTA: Ethylene Diamine Tetraacetic Acid
AESSM: Alkaline Ethanol Stop Solution Mix
Tris-HCl buffer: Tris(hydroxymethyl)aminomethane-HCl buffer
β-NADH: β-Nicotinamide Adenine Dinucleotide
EMEM: Eagle's Minimum Essential Medium
Sf9 cells: Clonal isolate, derived from Spodoptera frugiperda
HepG2 Cells: Human liver hepatocellular carcinoma cell line

### In Vitro Assay

### Example 8

hDGAT-1 Assay

The DGAT-1 assay was designed using human DGAT-1 enzyme over expressed in Sf9 cell-line as described in the reference, European Journal of Pharmacology, 650, 663-672, (2011), the disclosure of which is incorporated by reference for the teaching of the assay.

Cloning and Expression of Human DGAT-1 (hDGAT-1) Clone

hDGAT-1 ORF expression clone (RZPD0839C09146 in pDEST vector) was obtained from RZPD, Germany, hDGAT-1 gene (NM_{—}012079,) was cloned into pDESTS vector under strong polyhedron promoter of the Autographa californica nuclear polyhedrosis virus (AcNPV) with ampicillin resistance marker. The recombinant plasmid was introduced into DH10BAC competent cells (Invitrogen, US) by transformation which contains baculovirus shuttle vector (bacmid), and the resultant cells were streaked on to Luria broth (LB) agar plate containing ampicillin (100 μg/mL), kanaraycin (50 μg/mL) and of gentamycin (10 μg/mL) according to the Bac-to-Bac baculovirus Expression System (Invitrogen, US). The white colonies were picked and restreaked on to LB agar plates having above antibiotics and incubated overnight at 37° C. On the following day isolated white colonies with recombinant bacmid containing hDGAT-1 gene were inoculated into 10 mL of Luria broth with antibiotics (ampicillin (100 μg/mL), kanamycin (50 μg/mL) and gentamycin (10 μg/mL)) and incubated overnight with 200 rpm at 37° C. in an orbital shaker (New Brunswick). 10 mL of Luria broth was taken and recombinant bacmid DNA (with hDGAT-1 gene) was prepared using the Qiagen mini prep kit and was quantified using nanodrop. The concentration of the bacmid DNA containing hDGAT-1 gene was approximately 97 ng/μL.

Transfection and Virus Amplification using Sf9 Cells

1-3 μg of hDGAT-1 bacmid DNA was transfected into Sf9 cells using Cellfectin (Invitrogen, US) according to manufacturer's specifications in 6-well tissue culture plates. Transfected Sf9 cells were incubated at 27° C. for 5 h in incomplete Grace's insect media (Gibco®) without fetal bovine serum and antibiotic-antimycotic (100 units/mL), penicillin, (100 μg/mL), streptomycin sulphate, (0.25 μg/ml,) and amphotericin B. After completion of incubation media was replaced by growth media (Grace's insect media; (Gibco®) containing 10% fetal bovine serum (Hyclone) and antibiotic-antmycotic (100 units/mL), penicillin (100 μg/mL), streptomycin sulphate (0.25 μg/mL) and amphotericin B) and the cells were further incubated for 120 h at 27° C. in an incubator.

During this incubation, viral particles formed within the insect cells and were secreted. The supernatant containing the virus was collected at the end of 120 h by centrifuging at 1500×g for 5 min using Biofuge statos centrifuge (Heraeus 400), and was filtered through 0.22 μm filter (Millipore). It was stored as P1 recombinant baculovirus at 4° C. The cont >10⁵ pfu (plaque forming units)/mL were determined by the plaque assay conducted as per manufacturer's protocol (Invitrogen kit).

P1 recombinant baculovirus was further amplified at a MOI (multiplicity of infection) of 0.05-0.1, to generate P2 recombinant baculo virus in T-25 flask (Nunc) containing 5×10⁶ Sf9 cells in 5 mL complete Grace's insect media for 120 h followed by centrifugation at 1500×g for 5 min, filtration through 0.22 μm filter (Millipore), and storage at 4° C. as P2/(>106 pfu/mL) recombinant baculoviras. Similarly P3 and P4 recombinant baculovirus was further amplified, by reinfection at a MOI of 0.05-0.1, to generate P3 and P4 recombinant baculovirus respectively and were stored at 4° C. until further use. Viral titer for the P4 recombinant baculovirus was determined and it was found to be 1×10⁸ pfu/mL. The P4 (>10⁸ pfu/mL) recombinant baculo virus was finally used to infect sf9 cells at a MOI of 5-10.

### Microsome Preparation

Sf9 cells (2×10⁶ Cells/mL) grown in a 500 mL spinner flask containing 250 mL of Grace's insect cell media (Gibco) with antibiotic-antimycotic (Gibco®) and were infected with hDGAT-1 recombinant baculovirus (25 mL) at an MOI of 5. The infected ceils were maintained for 48 h at 28° C. and the cell pellet was collected by centrifuging the media at 1000×g at room temperature. The pellet was washed with PBS (pH 7.4) to eliminate residual media.

Cells were then disrupted by suspending the pellet in 15 mL of microsome preparation buffer containing 1×amount of protease cocktail tablet (Roche) and in house prepared protease inhibitor mixture by passing the lysate through a 27G needle followed by mild sonication at 4° C. The ceil debris was separated and the post nuclear supernatant (PNS), the lysate was centrifuged at 1000×g for 10 min at 4° C. using Biofuge statos centrifuge (Heraeus 400). The PNS obtained was then centrifuged at 15000×g for 30 min at 4° C. using the Biofuge statos centrifuge (Heraeus) to separate the post mitochondrial supernatant (PMS). Finally, uitracentrifugation was done at 100,000×g for 1 h at 4° C. using BeckmaTi-rotor to obtain microsomal pellet. To increase purity, the pellet was washed two times in microsomal preparation buffer containing in house preparation of a protease inhibitor mixture (Aprotinin (0.8 μM), pepstatin A (10 μM) and leupeptin (20 μM)-Sigma).

Finally microsomal pellet was suspended in 1.5 mL of the microsome preparation buffer and protein concentration was determined by Bradford method.

The microsomes were stored as aliquots of 100 μL each at −70° C. for in vitro assay.

### Preparation of Buffers and Reagents

### Stock Solutions

hDGAT-1 assay buffer stock: Assay buffer of pH 7.4 was prepared by dissolving 0.25 M sucrose (Sigma) and 1 mM EDTA (Sigma) in 150 mM tris HQ (Sigma).

Stop solution: For making 10 mL of Stop solution, 7.84 ml, of isopropanol (Qualigens) and 1.96 mL of n-heptane (Qualigens) were added in 0.2 mL de-ionized water.

A.E.S.S.M (alkaline ethanol stop solution mix): For making 10 mL of A.E.S.S.M solution, 1.25 mL of denatured ethanol, 1.0 mL of de-ionized water, and 0.25 mL of IN NaOH (Qualigens) were added to 7.5 mL of Stop solution.

Scintillation fluid: For making 2.5 L of scintillating fluid, 1667 mL toluene (Merck), 833 mL triton X-100 (Sigma), 12.5 g 2,5-diphenyloxazole (PPO; Sigma) and 500 mg (1,4-bis (5-phenyl-2-oxazolyl) benzene (POPOP; Sigma) were mixed.

### Working Stock

hDGAT-1 assay buffer: Freshly hDGAT-1 assay buffer containing 0.125% of BSA (free fatty acid, Sigma) was prepared before use.

Substrate mix preparation: Substrate mix was freshly prepared by adding 2047.5 μM of 1,2-dioleoyl-sn-glycerol (19.5 mM; Sigma) and 280 nCi/mL of [¹⁴C]oleoyl-CoA (0.1 mCi American Radiolabeled Chemicals/mL) and the final volume was made up to 1000 μL using hDGAT-1 assay buffer.

hDGAT-1 Enzyme preparation: Enzyme was diluted to a working concentration of 1 mg/mL in hDGAT-1 assay buffer, 2.5 μL of the working enzyme stock was used in hDGAT-1 assay (final concentration 25 μg/mL).

### Preparation of Test Samples

The test samples were prepared as follows. A stock solution of 20 mg/mL was prepared for each extract (Extract of Example 1 to Extract of Example 7) in 100% dimethyl sulfoxide (DMSO). The working stock was prepared in hDGAT-1 assay buffer. 10 μL of working stock was added into 100 μL of assay mixture to obtain the final concentration of extracts at 50 μg/mL.

Three different concentrations for dose response (i.e. 25 μg/mL, 50 μ/mL and 100 μg/mL) were prepared for Extract of Example 1 by serial dilution of stock solution.

### Assay

60 μL of substrate mix (as described above) was added to a total assay volume of 100 μL. The reaction was started by adding 2.5 μg hDGAT-1 containing microsomal protein and was incubated at 37° C. for 10 min. The reaction was stopped by adding 300 μL of alkaline ethanol stop solution mix (AESSM). The reaction involves the incorporation of radioactive [¹⁴C] oleoyl-CoA into the third hydroxyl group (OH) of 1,2-dioleoyl-sn-glycerol to form the radioactive triglyceride ([¹⁴C] triglyceride) which was then extracted into the upper heptane phase. The radioactive triglyceride product thus formed was separated into the organic phase by adding 600 μL of n-heptane. 250 μL of the upper heptane was added into 4 mL of scintillation fluid and measured using a liquid scintillation counter (Packard; 1600CA) as disintegration per min (dpm) counts. The percentage inhibition was calculated with respect to the vehicle. Results are -presented in Table 1.

The dose response was determined at concentrations of 25 μg/mL, 50 μg/mL and 100 μg/mL by serially diluting stock solution of Extract of Example 1 in hDGAT-1 assay buffer. Results are presented in Table 2.

TABLE 1
hDGAT-1 inhibition of extracts

| | | | | % inhibition |
|---|---|---|---|---|
| Sr. No. | Sample | Concentration. | | of hDGAT-1 |
| 01 | Extract of Example 1 | 50 | μg/mL | 72.15 |
| 02 | Extract of Example 2 | 50 | μg/mL | 85.81 |
| 03 | Extract of Example 3 | 50 | μg/mL | 82.37 |
| 04 | Extract of Example 4 | 50 | μg/mL | 85.84 |
| 05 | Extract of Example 5 | 50 | μg/mL | 42.04 |
| 06 | Extract of Example 6 | 50 | μg/mL | 16.07 |
| 07 | Extract of Example 7 | 50 | μg/mL | 48.50 |
| 08 | IN 5530* | 20 | nM | 46.27 |
| 09 | IN 5530* | 0.1 | μM | 66.04 |

*IN5530: In-house standard compound (2-((1s,4s)-4-(4-(7,7-dimethyl-7H-pyrimido [4,5-b][1,4]oxazin-6-yl)phenyl)cyclohexyl)acetic acid).

Conclusion: The extracts of the plant Calophyllum inophyllum (Extracts of Example 1-5 and Extract of Example 7) were found to be active in the hDGTA-1 inhibition assay.

TABLE 2
Dose- response of Extract of Example 1 in hDGAT-1 inhibition assay

| | | | | % Inhibition |
|---|---|---|---|---|
| Sr. No. | Sample | Concentration | | of hDGAT-1 |
| 01 | Extract of Example 1 | 25 | μg/mL | 79.04 |
| 02 | Extract of Example 1 | 50 | μg/mL | 87.66 |
| 03 | Extract of Example 1 | 100 | μg/mL | 90.48 |
| 04 | IN5530 (Std. com.) | 20 | nM | 48.82 |
| 05 | IN5530 (Std. com.) | 0.1 | μM | 74.26 |

*IN5530: In-house standard compound (2-((1s,4s)-4-(4-(7,7-dimethyl-7H-pyrimido [4,5-b][1,4]oxazin-6-yl)phenyl)cyclohexyl)acetic acid).

Conclusion: Extract of Example 1 showed dose-related activity in the hDGAT-1 inhibition assay.

### Example 9

### SCD-1 Assay

The assay was carried out according to the method described in reference, European Journal of Pharmacology, 618, 28-36, (2009), the disclosure of which is incorporated by reference for the teaching of the assay.

Preparation of SCD-1 Enzyme

The SCD-1 enzyme was prepared from rat liver microsomes as described in PCT Publication Application WO2008/074835A1, the disclosure of which is incorporated by reference for the teaching of the assay.

Male Sprague-Dawley rats (150-175 g) were fasted for two days and then fed on low fat diet for three days to induce SCD-1 activity. The rats were then sacrificed and their livers were removed and placed on ice. The livers were finely chopped with scissors and then homogenized using a Polytron homogenizer in a homogenization buffer (150 mM KCl, 250 mM sucrose, 50 mM tris-HCl, pH 7.5, 5 mM EDTA, and 1.5 mM reduced glutathione) at 4° C. The homogenate was centrifuged at 1500×g for 20 min at 4° C. The supernatant was collected and centrifuged twice at 10,000×g for 20 min each at 4° C. The resultant supernatant was collected and centrifuged at 100,000×g for 60 min at 4° C. The supernatant was discarded and the microsomal pellet was resuspended in homogenization buffer, aliquoted, and stored at −80° C. The protein content of the resuspended pellet was identified by Bradford assay.

### Preparation of Buffers and Reagents

Preparation of SCD-1 assay buffer: The buffer consisted of 100 mM K₂HPO₄ (Qualigens) and 100 mM Na₂H₂PO₄.2H₂O (Qualigens), pH 7.4.

Preparation of potassium phosphate buffer: The buffer consisted of 200 mM K₂HPO₄(Qualigens), and 200 mM KH₂PO₄ (Qualigens), pH 7.0.

Preparation of SCD-1 extraction buffer: The buffer consisted of 250 mM sucrose (Sigma), 15 mM N-acetyl cysteine (Sigma), 5 mM MgCl (Sigma), 0.1 mM EDTA (Sigma), 0.15 M KCl (Sigma), and potassium phosphate buffer 62 mM, pH 7.0.

Preparation of P-NADH: A 20 mM stock solution of β-NADH (Sigma) was prepared in SCD-1 assay buffer and stored at −70° C. Working stock of β-NADH was prepared by diluting the stock to 8 mM with assay buffer just before use.

Preparation of stearoyl co-A: A 1.65 mM stock solution of stearoyl co-A (Sigma) was prepared in SCD-1 assay buffer and stored at −70° C.

Preparation of radioactive cocktail: 100 μL of 1 μCi/mL stearoyl (9,10³H) Co A (American Radiolabeled Chemicals) and 144 μL of 1.65 mM stearoyl co-A was added to 5516 μL of SCD-1 assay buffer.

### Preparation of Activated Charcoal Beds in a Multiscreen Plates

A 33% activated charcoal (Sigma) solution was made in assay buffer. 250 μL of the solution was added to each well of a multiscreen plate. The charcoal bed was formed by applying vacuum to the plate through a vacuum manifold. The plates were stored till use.

### Preparation of Test Samples

The test samples were prepared as follows. A stock solution of 20 mg/mL was prepared for each extract (Extract of Example 1 to Extract of Example 4) in 100% dimethyl sulfoxide (DMSO). The working stock was prepared in SCD-1 assay buffer. 10 μL of working stock was added into 100 μL of assay mixture to obtain the final concentration of extracts at 50 μg/mL.

### Assay

The microsomes (62.5 μg) were treated with the test sample for 15 min. After which 25 μL β-NADH working stock and 20 μL of radioactive cocktail containing 9,10-³H stearoyl CoA were added and the mixture was incubated at 25° C. for 30 min. The reaction was terminated by the addition of perchloric acid. The plate was then centrifuged and the supernatant from each well passed through charcoal beds into reservoir plates using the vacuum manifold. The filtrate containing ³H₂O was transferred to scintillation vials containing 4 mL of scintillation fluid and the cpm counts were measured using a liquid scintillation counter. The % inhibition was calculated with reference to the vehicle control. A positive control was also assayed with each experiment. Results are presented in Table 3.

TABLE 3
SCD-1 inhibition of extracts of Calophyllum inophyllum

| Sr. No. | Sample | Concentration | | % inhibition of SCD-1 |
|---|---|---|---|---|
| 01 | Extract of Example 1 | 50 | μg/mL | 56.7 |
| 02 | Extract of Example 2 | 50 | μg/mL | 63.6 |
| 03 | Extract of Example 3 | 50 | μg/mL | 65.7 |
| 04 | Extract of Example 4 | 50 | μg/mL | 68.0 |
| 05 | MF-152* | 100 | nM | 56.1 |

*MF-152: Standard compound [Bioorganic & Medicinal Chemistry Letters, 19, 5214-5217, (2009)].

Conclusion: The extracts (Extracts of Example 1 to Extract of Example 4) of Calophyllum inophyllum were found to be active in the SCD-1 inhibition assay.

### Example 10

### Cell Based Triglyceride (TG) Synthesis Assay

The Extract of Example 1 selected from the primary assays was evaluated for it's ability to inhibit triglyceride synthesis in HepG2 cells by the method as reported in reference, European Journal of Pharmacology, 618, 28-36, (2009), the disclosure of which is incorporated by reference for the teaching of the assay.

### Preparation of Buffers, Reagents and Media

Eagle's minimum essential medium (EMEM): One sachet of powdered EMEM (Sigma) was added to a 1 L conical flask. The empty sachet was rinsed with 10 mL of distilled water. The powder was dissolved in 900 mL distilled water using a magnetic stirrer. 1.5 g sodium bicarbonate (Sigma), 10 mL sodium pyruvate (Sigma) and 1 mL of Penicillin-Streptomycin (Gibco) was also supplemented. After proper mixing the pH was adjusted to 7.2. and the volume made up to 1 L. The medium was filter sterilized and was stored at 4° C.

Inactivated fetal bovine serum (FBS): Fetal bovine serum (Hyclone) was placed in a water-bath preset at 56° C. for 30 min. The FBS was then aliquoted (45 ml.) in 50 ml. polypropylene tubes and was stored at −80° C.

Phosphate buffered saline (PBS): Contents of one sachet of PBS (Sigma) were dissolved in 900 mL of distilled water. The pH was adjusted to 7.2 and the volume made up to 1 L. It was then filtered sterilized and was stored at −20° C.

Trypsin-EDTA solution: Trypsin-EDTA solution (Sigma) was thawed and aseptically aliquoted (45 mL) in 50 mL polypropylene tubes and was stored at −20° C.

### Preparation of Test Samples

The test samples were prepared as follows. A stock solution of 20 mg/mL was prepared for the Extract of Example 1, in 100% dimethyl sulfoxide (DMSO). 10 μL of working stock was added into 100 μL of assay mixture to obtain the final concentration of extracts at 50 μg/mL.

Three different concentrations for dose response (i.e. 25 μg/mL, 50 μg/mL and 100 μg/mL) were prepared for Extract of Example 1 by serial dilution of stock solution.

### Culturing of HepG2 Cells

One frozen vial of HepG2 cells (ATCC No. HB-8065) was thawed in water at 37° C. All the contents of the vial were transferred into a T-75 tissue culture flask containing 9 mL of EMEM and 1 mL inactivated fetal bovine serum. The flask was incubated at 37° C., with 5% CO₂ in a humidity controlled incubator. The flasks were observed for cell growth. When the cells were −70% confluent the spent medium was discarded and the cell monolayer was washed with 5 mL of PBS. 1.5-2 mL of Trypsin EDTA solution was added to the flask such that the entire cell layer was covered. When all the cells from the flask were detached, 6 mL of EMEM supplemented with 10% fetal bovine serum was added and mixed to get a uniform cell suspension. The cell suspension was centrifuged at 1000 rpm for 5 min to obtain a pellet of cells. The cell pellet was gently dispersed in 6 mL of EMEM supplemented with 10% fetal bovine serum. Six T-75 flasks were prepared as described above and 1 mL of the cell suspension was added to each of the flasks. The flasks were incubated for 24 h at 37° C. with 5% CO₂ in a humidity controlled incubator. The medium was changed after every 48 h. By 72 h the flasks were ˜70% confluent and ready for plating.

### Assay

A suspension of HepG2 cells was prepared in EMEM medium containing 10% fetal bovine serum. The cell count was determined using a haemocytometer and the count was adjusted to 4×10⁵ cells/mL/well for a 24 well plate. A parallel plate was also made for viability testing to be done at the end of the experiment. The plates were incubated at 37° C. with 5% CO, in a humidity controlled incubator till the cells were confluent. When the cells were 70-80% confluent, the medium was discarded and replaced with fresh medium containing the standard compound (ME-152) at 10 μM or Extract of example 1 at 50 μg/mL. DMSO was added in vehicle wells at a final concentration of 0.1%. The plates were incubated overnight for ˜18 h. Next day the medium was discarded and replaced with one containing standard compound/extract/DMSO supplemented with 0.1% BSA (fatty acid free).

2 μCi of ¹⁴C labeled acetic acid was also added per well and the plates were further incubated for 6 h at 37° C. after which the medium was discarded and lipids were extracted.

To assess the cytotoxic effects of the plant extracts, the cellular viability test was performed on the parallel plate using MTS (3-(4,5-dimethylthiazol-2-yl)-5-(3-carboxymethoxyphenyl)-2-(4-sulfonyl)-2H-tetrazolium) reagent after 2 h of incubation.

### Lipid Extraction

The extraction was carried out as per the following protocol:

At the end of the experiment, the cells were washed twice with ice-cold PBS. The cells were scrapped into 1 mL cold PBS and pipetted into 15 mL glass tubes containing 4 mL methanol xhioro form (2:1) and was stirred using vortex mixer. The tubes were spun at 4000 rpm for 5 min, and the supernatant was transferred into a new tube. The pellet consisting mostly of proteins was discarded. 1 mL of 50 mM citric acid, 2 mL of water and 1 ml, of chloroform was added to the above supematent and was stirred using vortex mixer. A turbid two phase mixture was obtained. The tubes were spun at 3500 rpm in a non-cooled centrifuge for 15 min. A lower chloroform phase and an upper water/methanol phase were obtained. There was also an inter-phase between the two that consists mostly of precipitated protein. The upper water/methanol phase was discarded, leaving the inter-phase untouched. The lower chloroform phase containing the lipids was transferred into a new tube, and was evaporated on a heating block. The lipids were re-dissolved in 200 μL of chloroform:methanol (2:1). The triglycerides were isolated on TLC silica plates using a solvent system of hexane:diethylether:acetic acid (85:15:0.5). A non-radiolabelled triglyceride standard was run alongside as well as all spots were co-spotted with triglyceride standard. The TLC plates were exposed to iodine vapors and the triglyceride spots were scrapped off and transferred to scintillation vials containing 4 mL of scintillation fluid. The radioactivity was measured in cpm in a liquid scintillation counter and the inhibition was calculated with reference to the vehicle. Results are presented in Table 4.

The dose response was determined at concentrations of 25 μg/mL, 50 μg/mL and 100 μg/mL by serially diluting stock solution of Extract of Example 1. Results are presented in Table 5.

TABLE 4
Inhibition of triglyceridesynthesis by Extract of Example 1

| Sr. No. | Sample | Concentration | | % Inhibition of Tg |
|---|---|---|---|---|
| 01 | Extract of Example 1 | 50 | μg/mL | 69.65 |
| 02 | MF-152 | 1.0 | μM | 34.19 |

*MF-152: Standard compound (Bioorganic & Medicinal Chemistry Letters, 19, 5214-5217, (2009)).

Conclusion: Extract of Example 1 was found to be active in the cell based triglyceride synthesis assay.

TABLE 5
Inhibition of Triglyceride synthesis by Extract of Example 1

| | | | | % inhibition of | % |
|---|---|---|---|---|---|
| Sr. No. | Sample | Concentration | | Triglyceride | Toxicity |
| 01 | Extract of Example 1 | 25 | μg/mL | 31.12 | 12 |
| 02 | Extract of Example 1 | 50 | μg/mL | 55.37 | 46 |
| 03 | Extract of Example 1 | 100 | μg/mL | 84.12 | 66 |
| 04 | MF-152 | 10 | μM | 35.13 | 0 |

*MF-152: Standard compound (Bioorganic & Medicinal Chemistry Letters, 19, 5214-5217, (2009)).

Conclusion: Extract of Example 1 showed dose-related inhibition of triglyceride synthesis.

### In Vivo Study

The in vivo experiments were carried out in accordance with the guidelines of Committee for the Purpose of Control and Supervision of Experiments on Animals (CPCSEA) and with the approval of Institutional Animal Ethics Committee (IAEC).

### Example 11

### Effect of Extract of Example 1 on High Fat Diet (HFD)-Induced Body Weight Gain

The high fat diet (HFD) induced obesity model in rodents has been reported to be a useful model for evaluating the efficacy of anti-obesity agents (Obesity, 17(12), 2127-2133, (2009)). It has been reported that feeding a high-fat diet containing 58% kcal fat caused obesity in mice (Metabolism, 47, 1354-1359, (1998)). In addition, the mice fed on the high-fat diet has shown significantly higher body weight and significantly heavier visceral adipose tissues (e.g., epididymal, retroperitoneal and mesenteric adipose tissues) than the mice which were fed on the normal diet (Life Sciences, 77, 194-204, (2005)).

The HFD induced body weight gain model was reported for evaluating the anti-obesity effects of various natural products (BMC Complementary and Alternative Medicine, 5:9, 1-10, (2005); BMC Complementary and Alternative Medicine, 6:9, 1-9, (2006)).

A HFD induced body weight gain study in mice was conducted to evaluate the efficacy of the Extract of Example 1.

Male C57BL/6J mice (in-house; Central Animal Facility, Piramal Healthcare Limited, Goregaon, Mumbai, Maharashtra, India) were acclimatized with HFD (60% Kcal, D12492, Research Diets, USA) for two weeks. Mice exhibiting weight gain were selected for the study and were randomized into treatment groups consisting of 10 mice each.

### Preparation of Test Sample

A suspension of Extract of Example 1 was prepared in polyethyleneglycol 400 (30%) (PEG 400, Fisher Scientific, India) and 0.5% carboxy methylcellulose (70%) (CMC, Sigma, USA).

### Assay

The Extract of Example 1 was administered at a dose of 500 mg/kg body-weight orally, once daily. Orlistat (Biocon, India) was used as the standard drug and was administered orally at a dose of 15 mg/kg body weight, twice daily. A separate group of ten mice was fed a low fat diet (LED, 10% kcal, D12450B, Research Diet, USA) as a normal control. Vehicle was administered to the HFD and LFD control groups at dose of 10 mL/kg body weight.

The treatments were continued for a period of sixty days. Body weight and feed intake were monitored daily. The % change in body weight (% increase in body weight from day 1) and the cumulative feed intake data was calculated. On day sixtyone, blood samples (˜200 μL/mice) were collected in heparinised (50 IU/mL) micro-centrifuge tubes under isoflurane anesthesia. Plasma was separated by centrifugation at 10000 rpm at 4° C. for estimation of various plasma biochemistry parameters. The biochemistry analysis was performed on BS-400 autoanalyzer (Mindray, China). Subsequently, the mice were sacrificed and following organs/tissues were dissected out and weighed viz., liver, heart, kidneys, epididymal fat and retroperitoneal fat. All the data was analyzed for statistical significance by one-way ANOVA followed by Dunnet's post-hoc test and values of P<0.05 were considered as significant. All analyses were carried out using GraphPad Prism version 4.00 for Windows (GraphPad Software, San Diego, Calif., USA). Results are presented in Table 6, Table 7 and Table 8.

TABLE 6
Effect of Extract of Example 1 on HFD induced body weight gain in mice

| | Body Weight (g) | Body Weight (g) | % Change in |
|---|---|---|---|
| Group | (Day 0) | (Day 60) | body weight |
| LFD + Vehicle | 24.8 ± 0.7 | 27.1 ± 0.6 | 9.66 ± 1.56** |
| HFD + Vehicle | 26.5 ± 0.6 | 32.9 ± 1.0 | 24.28 ± 1.15 |
| HFD + Extract of | 26.5 ± 0.6 | 30.6 ± 1.4 | 14.10 ± 3.57* |
| Example 1 | | | |
| HFD + Orlistat | 26.4 ± 0.4 | 30.9 ± 0.9 | 16.74 ± 2.70 |

*p < 0.05,
**p < 0.01 Vs. HFD + Vehicle;
Mean ± S.E.M.

The Extract of Example 1 showed significant inhibition of body weight gain as compared to HFD+Vehicle group.

TABLE 7
Effect of Extract of Example 1 on cumulative feed intake

| | Cumulative Feed intake (g/mice) |
|---|---|
| Group | (Day 60) |
| LFD + Vehicle | 120.3 ± 3.3 |
| HFD + Vehicle | 110.5 ± 2.8 |
| HFD + Extract of Example 1 | 105.8 ± 3.1 |
| HFD + Orlistat | 119.7 ± 4.7 |

Mean ± S.E.M.

No significant reduction in cumulative feed intake was observed in the Extract of Example 1 when compared to the HFD+Vehicle group.

TABLE 8
Effect of Extract of Example 1 on adipose tissue weight

| | Epididymal | Retroperitoneal | Total Fat ^{#} |
|---|---|---|---|
| Group | Fat (g) | Fat (g) | (g) |
| LFD + Vehicle | 0.43 ± .03** | 0.17 ± 0.01** | 0.60 ± 0.05** |
| HFD + Vehicle | 1.39 ± 0.13 | 0.65 ± 0.08 | 2.03 ± 0.20 |
| HFD + Extract of Example 1 | 1.03 ± 0.15 | 0.45 ± 0.08 | 1.48 ± 0.23 |
| HFD + Orlistat | 1.07 ± 0.10 | 0.42 ± 0.05* | 1.49 ± 0.15 |

^{#} Total fat = Epididymal fat + Retroperitoneal fat,
*p < 0.05,
**p < 0.01 Vs. HFD + Vehicle;
Mean ± S.E.M.

Extract of Example 1 showed trend towards reduction in adipose tissue weight as compared to the HFD+vehicle group.

The plasma biochemistry analysis for parameters like glucose, triglyceride, cholesterol, alanine aminotransferase, aspartate aminotransferase, albumin, creatinine and urea showed no significant difference between Extract of Example 1 and the vehicle group. The organ weights (heart, liver and kidney) did not show any significant difference.

Conclusion: The treatment of Extract of Example 1 to mice on HFD caused significant reduction of body weight gain. This reduction in body weight gain was achieved without significant reduction in feed intake and was also evident in the reduced adipose tissue weight (fat mass). Extract Example 1 has shown antiobesity activity in the high fat diet (HFD) induced obesity model.

1-18. (canceled)

19. A composition comprising a therapeutically affective amount of an extract obtained from the plant Calophyllum inophyllum as an active ingredient along with at least one pharmaceutically acceptable carrier.

20. The composition as claimed in claim 19, wherein the composition contains 5-100% of extract of the plant Calophyllum inophyllum.

21. The composition as claimed in claim 19, wherein the extract is obtained from the bark of the plant Calophyllum inophyllum.

22. The composition as claimed in claim 19, wherein the extract of Calophyllum inophyllum contains a bioactive marker.

23. The composition as claimed in claim 19, wherein the composition is administered orally to a subject in need of the treatment for a metabolic disorder.

24. The composition as claimed in claim 23, wherein the composition is formulated for oral administration in the form of a tablet, capsule or granules.

25. A method of treating a metabolic disorder in a subject comprising administering to the subject a therapeutically effective amount of the composition as claimed in claim 19.

26. The method of claim 25, wherein the said composition is administered orally.

27. The method of claim 25, wherein the metabolic disorder is selected from insulin resistance, hyperglycemia type 2 diabetes, obesity, glucose intolerance, hypercholesterolemia, dyslipidemia, hyperinsulinemia, atherosclerotic disease, polycystic ovary syndrome, coronary artery disease, metabolic syndrome, hypertension or a related disorder associated with abnormal plasma lipoprotein, triglycerides or a disorder related to glucose levels.

28. The method of claim 27, wherein me metabolic disorder is type 2 diabetes.

29. The method of claim 27, wherein the metabolic disorder is obesity.

30. A method of treating a metabolic disorder in a subject comprising administering to the subject a therapeutically effective amount of the composition as claimed in claim 19 in combination with a therapeutically active agent selected from orlistat, pioglitazone, rosiglitazone, glibenclamide, glipizide, glimeperide, repaglinide, nateglinide or metformin.
